(19) United States
(12) Patent Application Publication
(10) Pub. No.: US 2009/0062337 A1
(43) Pub. Date: Mar. 5, 2009

(54) Treatment of Microbial Infections

(75) Inventors: William R. Schwan, Onalaska, WI (US); Aaron Monte, La Crosse, WI (US); Marc A. Rott, Victory, WI (US); Thomas J. Volk, La Crosse, WI (US)

Correspondence Address: Don D. Cha, 547 Buena Vista Road, Golden, CO, 80401, US

(73) Assignee: WYSIS TECHNOLOGY FOUNDATION, INC., Madison, WI (US)

(21) Appl. No.: 12/199,888

(22) Filed: Aug. 28, 2008

Related U.S. Application Data
(60) Provisional application No. 60/969,239, filed on Aug. 31, 2007.

(51) Int. Cl. A61K 31/4704; A61K 31/4706; A61P 31/04; A61P 31/10; A61P 31/18

(52) U.S. Cl. 514/312; 514/313

(57) ABSTRACT

The present invention provides methods for treating various microbial infections using a compound of the formula:

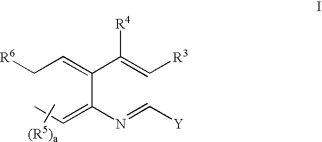

or an isomer, tautomer, prodrug or a pharmaceutically acceptable salt thereof, wherein Y, R³, R⁴, R⁵, R⁶, and a are those defined herein.

### CROSS-REFERENCE TO RELATED APPLICATIONS

This application claims the priority benefit of U.S. Provisional Application No. 60/969,239, filed Aug. 31, 2007, which is incorporated herein by reference in its entirety.

### FIELD OF THE INVENTION

The present invention relates to methods for treating bacterial and fungal infections. In particular, methods of the invention utilize 2-aminoquinoline and its derivatives.

### BACKGROUND OF THE INVENTION

While many antimicrobial compounds are available today, some microbial infections cannot be treated with the current antimicrobial compounds. This is particularly true in many patients who have other clinical conditions. For example, cystic fibrosis is caused by the defect of a gene coding for cystic fibrosis transmembrane conductance regulator (CFTR) protein and in the event of bacterial (e.g., Pseudonomas aeruginosa) infection of the respiratory organ leads to collection of mucous in the lungs and causes an obstruction of airways leading to premature death. In fact, several bacterial species are responsible for chronic infections and early mortality in cystic fibrosis (CF) patients. Some of these species are quite resistant to a variety of antibiotics, so treatment options have become more limited in patients colonized with these bacterial species. Though antibiotics and digestive enzymes are currently in use for the treatment of this disease, an adequate therapeutic response has yet to be achieved. Despite the need for new antibiotics, there are few new antimicrobials in the pipelines of pharmaceutical companies.

Therefore, there remains a continuing need for new methods for treating various microbial infections.

### SUMMARY OF THE INVENTION

Some aspects of the invention provide methods and compositions for treating various microbial infections in a subject. Many embodiments of the invention are directed to treating microbial infection in a subject who has other clinical conditions.

One particular aspect of the invention provides a method for treating bacterial infection in a subject comprising administering to the subject in need of such treatment a compound of the formula:

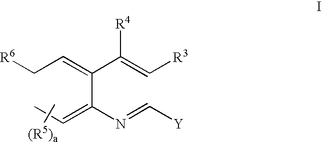

or an isomer, tautomer, prodrug or a pharmaceutically acceptable salt thereof,
wherein
Y is —NR¹R², —OR⁷, or —SR^{7;}
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and

wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷;
R⁶ is selected from the group consisting of hydrogen, halogen, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ—NR⁷CO₂R⁷, —(CH₂)ₙ—NR⁷C(O)N(R⁷)₂, —(CH₂)ₙ—NR⁷SO₂R⁷, —(CH₂)ₙ—S(O)₂R⁷, —(CH₂)ₙ—SO₂N(R⁷)₂, —(CH₂)ₙ—OR⁷, —(CH₂)ₙ—OC(O)R⁷, —(CH₂)ₙ—OC(O)OR⁷, —(CH₂)ₙ—OC(O)N(R⁷)₂, —(CH₂)ₙ—N(R⁷)₂, and —(CH₂)_{—NR}⁷SO₂N(R⁷)₂, wherein one or two of the hydrogen atoms in (CH₂)ₙ may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, C(O)N(R^{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C⁰⁻⁴ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₀₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

In some embodiments of the invention, the bacterial infection comprises lung infection, wound infection, urinary tract infection or a combination thereof. Within these embodiments, in some instances the bacterial infection is in the lung of cystic fibrosis patient.

Often, the bacterial infection is caused by a gram negative bacteria.

In other embodiments, the bacterial infection is caused by bacteria comprising Burkholderia cepacia, Pseudomonas aeruginosa, Stenotrophomonas maltophilia, A. xylosoxidans, A. baumannii, or a combination thereof.

Another particular aspect of the invention provides a method for treating bacterial infection caused by a bacteria comprising Burkholderia cepacia, Pseudomonas aeruginosa, and Stenotrophomonas maltophilia, A. xylosoxidans, A. baumannii, or a combination thereof in a subject by, said method comprising administering to the subject in need of such treatment a compound of Formula I.

In some embodiments, the bacteria infection comprises lung infection, wound infection, urinary tract infection or a combination thereof.

In other embodiments, the bacterial infection comprises lung infection of a cystic fibrosis patient.

Another aspect of the invention provides a method for treating bacterial infection comprising lung infection, wound infection, urinary tract infection, or a combination thereof in a subject. The method generally comprises administering to the subject in need of such treatment a compound of Formula I.

In some embodiments, the subject has cystic fibrosis.

Still in other embodiments, the bacterial infection is caused by a bacteria comprising Burkholderia cepacia, Pseudomonas aeruginosa, and Stenotrophomonas maltophilia, A. xylosoxidans, A. baumannii, or a combination thereof.

Yet another aspect of the invention provides a method for treating fungal infection in a subject comprising administering to the subject in need of such treatment a compound Formula I.

In some embodiments, the fungal infection is caused by a fungus comprising Cryptococcus neoformans, Candida albicans, Aspergillus fumigatus, Trichophyton mentagrophytes, or a combination thereof. Within these embodiments, in some instances the subject has immunodeficiency. In some cases within these instances, the subject has AIDS.

### DETAILED DESCRIPTION OF THE INVENTION

### Definitions

Alkyl”, as well as other groups having the prefix “alk”, such as alkoxy, alkanoyl, means carbon chains which may be linear or branched or combinations thereof Examples of alkyl groups include methyl, ethyl, n-propyl, isopropyl, n-butyl, 1-methylpropyl, 2-methylpropyl, tert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethyl butyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like.

“Alkenyl” means carbon chains which contain at least one carbon-carbon double bond, and which may be linear or branched or combinations thereof Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, dihydroindanyl, 3,3-spirohexylindoline, 5,6,7,8-tetrahydroquinoline, and the like.

“Aryl” means mono- or bicyclic aromatic rings containing only carbon atoms. The term also includes aryl group fused to a monocyclic cycloalkyl or monocyclic heterocycloalkyl group in which the point of attachment is on the aromatic portion. Examples of aryl include phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one ring heteroatom selected from N, O and S, with each ring containing 5- to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, thienopyridinyl, and the like.

“Heterocycloalkyl” means mono- or bicyclic saturated rings containing at least one ring heteroatom selected from N, S and O, each of said ring having from 3 to 14 atoms in which the point of attachment may be carbon or nitrogen. The term also refers to bridged rings, includes monocyclic heterocycles fused to an aryl or heteroaryl group in which the point of attachment is on the non-aromatic portion, and also includes spirocyclic rings in which the point of attachment is via a heterocyclic ring. Examples of “heterocycloalkyl” include azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 2-oxa-5-azabicyclo[2.2.1]heptyl, 2,5-diazabicyclo[2.2.1]heptyl, 2-azabiclyclo[2.2.1]heptyl, 7-azabicyclo[2.2.1.]heptyl, 2,4-dizaobicyclo[2.2.2]octyl, 2-azabicyclo[2.2.2]octyl, 3-azabicyclo[3,2.2]nonyl, 2H-pyrrolyl, 4,4-spiro[2,3-dihydrobenzothiophen-3,3-yl]piperidinyl, 4,4-spiro[indoli-3,3-yl]piperidinyl, 2,7-diazaspiro[4.4]nonyl, 2,7-diazaspiro[4.5]decyl, 2,7-diazaspiro[4.6]undecyl, 1,7-diazaspiro[4.4]nonyl, 2,6-dizaospiro[4.5]decyl, 2,6-diazaspiro[4.6]-undecyl and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” includes fluorine, chlorine, bromine and iodine.

Some compounds of Formula I contain one or more asymmetric centers and can thus occur as racemates and racemic mixtures, enantiomerically enriched mixtures, single enantiomers, diastereomeric mixtures and individual diastereomers. It should be understood that the scope of the invention encompasses all such isomeric forms of the compounds of Formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixtures thereof are encompassed within the scope of the invention.

Compounds of the Formula I may be separated into diastereoisomeric pairs of enantiomers by, for example, fractional crystallization from a suitable solvent, for example MeOH or ethyl acetate or a mixture thereof. The pair of enantiomers thus obtained may be separated into individual stereoisomers by conventional means, for example, by the use of an optically active amine as a resolving agent or on a chiral HPLC column. Alternatively, any enantiomer of a compound of Formula I may be obtained by stereo specific synthesis using optically pure starting materials or reagents of known configuration.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Typical pharmaceutically acceptable acids are those made with citric, hydrobromic, hydrochloric, maleic, phosphoric, sulfuric, and tartaric acids.

It should be understood that, unless the context requires otherwise, references to the compounds of Formula I are meant to also include the pharmaceutically acceptable salts.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its mirror image.

The terms “halo,” “halogen” and “halide” are used interchangeably herein and refer to fluoro, chloro, bromo, or iodo.

“Enantiomeric excess” refers to the difference between the amount of enantiomers. The percentage of enantiomeric excess (% ee) can be calculated by subtracting the percentage of one enantiomer from the percentage of the other enantiomer. For example, if the % ee of (R)-enantiomer is 99% and % ee of (S)-enantiomer is 1%, the % ee of (R)-isomer is 99%-1% or 98%.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

The terms “pro-drug” and “prodrug” are used interchangeably herein and refer to any compound which releases an active parent drug according to Formula I in vivo when such prodrug is administered to a mammalian subject. Prodrugs of a compound of Formula I are prepared by modifying one or more functional group(s) present in the compound of Formula I in such a way that the modification(s) may be cleaved in vivo to release the parent compound. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I, and the like.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

As used herein, the term “treating” , “contacting” or “reacting” refers to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

It should be appreciated that when a variable is indicated ranging from zero (0) to “b” , e.g., cycloalkyl C_{0-b} alkyl, it means that alkyl group can be absent (i.e., when C is zero (C₀)), or it can be a chain of alkyl group where the number of carbons in the chain is defined by the subscript (i.e., from 1 to b).

### Compounds of the Invention

In one aspect, the invention provides methods for using a compound of the formula:

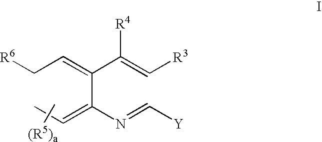

or an isomer, tautomer, prodrug or a pharmaceutically acceptable salt thereof,
wherein
Y is —NR¹R², —OR⁷, or —SR⁷;
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷;
R⁶ is selected from the group consisting of hydrogen, halogen, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ—NR⁷CO₂R⁷, —(CH₂)ₙ—NR⁷C(O)N(R⁷)₂, —(CH₂)ₙ—NR⁷SO₂R⁷, —(CH)ₙ—S(O)₂R⁷, —(CH₂)ₙ—SO₂N(R⁷)₂, —(CH₂)ₙ—OR⁷, —(CH₂)ₙOC(O)R, —(CH₂)ₙ—OC(O)OR⁷, —(CH₂)ₙ—OC(O)N(R⁷)₂, —(CH₂)ₙ—N(R⁷)₂, and —(CH₂)ₙ—NR⁷SO₂N(R⁷)₂, wherein one or two of the hydrogen atoms in (CH₂)ₙ may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R_{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C₀₋₄ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₀₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

It is to be understood that the scope of this invention encompasses not only the various tautomers (e.g., when R⁴ is —OH) and/or isomers that may exist but also the various mixture of tautomers and/or isomers that may be formed. Furthermore, the scope of the invention also encompasses solvates and salts of Compounds of Formula I.

In some embodiments, R¹ is selected from hydrogen, C₁₋₆ alkyl, C₂₋₆ alkenyl, C₂₋₆ alkynyl, cycloalkyl-C₀₋₆ alkyl, heterocycloalkyl-C₀₋₁₀ alkyl, aryl-C₁₋₁₀ alkyl, and heteroaryl-C₀₋₁₀ alkyl; wherein alkyl, alkenyl, and alkynyl, moieties are optionally substituted with one to four substituents independently selected from R^{a}, and cycloalkyl, heterocycloalkyl aryl and heteroaryl moieties above are optionally substituted with one to four substituents independently selected from R^{b}; and wherein sulfur-containing heterocyclic rings may be mono- or di-oxidized on the sulfur atom.

In some classes of these embodiments, R¹ is selected from hydrogen, C₁₋₆ alkyl, C₂₋₆ alkenyl, cycloalkyl-C₀₋₆ alkyl, heterocycloalkyl-C₀₋₆ alkyl, aryl-C₀₋₆ and heteroaryl-C₀₋₁₀ alkyl; wherein alkyl and alkenyl moieties are optionally substituted with one to three substituents independently selected from R^{a}, and cycloalkyl, heterocycloalkyl, aryl and heteroaryl moieties above are optionally substituted with one to three substituents independently selected from R^{b}.

In some subclasses of these classes, R¹ is hydrogen, or C₁₋₆ alkyl, optionally substituted with one to three substituents independently selected from R^{a}.

In other subclasses of these classes, R¹ is hydrogen, methyl, ethyl, or propyl, optionally substituted with one to three substituents independently selected from R^{a}.

In yet another subclasses of these classes, R¹ is hydrogen or methyl that is optionally substituted with one to three substituents independently selected from R^{a}.

In still other subclasses of these classes, R¹ is hydrogen or methyl.

In one embodiment of the present invention, R² is selected from: (1) hydrogen, (2) C₁₋₆ alkyl, (3) C₂₋₆ alkenyl, (4) C₂₋₆ alkynyl, (5) cycloalkyl-C₀₋₆ alkyl, (6) heterocycloalkyl-C₀₋₁₀ alkyl, (7) aryl-C₀₋₁₀ alkyl, and (8) heteroaryl-C₀₋₁₀ alkyl; wherein alkyl, alkenyl, and alkynyl, moieties above are optionally substituted with one to four substituents independently selected from R^{a}, and cycloalkyl, heterocycloalkyl aryl and heteroaryl moieties above are optionally substituted with one to four substituents independently selected from R^{b}; and wherein sulfur-containing heterocyclic rings may be mono- or di-oxidized on the sulfur atom.

In other embodiments, R² is hydrogen, C₁₋₆ alkyl, C₂₋₆ alkenyl, cycloalkyl-C₀₋₆ alkyl, heterocycloalkyl-C₀₋₆ alkyl, aryl-C₀₋₆ alkyl, or heteroaryl-C₀₋₁₀ alkyl; wherein alkyl and alkenyl moieties are optionally substituted with one to three substituents independently selected from R^{a}, and cycloalkyl, heterocycloalkyl, aryl and heteroaryl moieties are optionally substituted with one to three substituents independently selected from R^{b}.

In some subclasses of these classes, R² is hydrogen, C₁₋₆ alkyl, cycloalkyl-C₀₋₆ alkyl, heterocycloalkyl-C₀₋₆ alkyl, aryl-C₀₋₆ alkyl, or heteroaryl-C₀₋₁₀ alkyl; wherein alkyl moieties are optionally substituted with one to three substituents independently selected from R^{a}, and cycloalkyl, heterocycloalkyl, aryl and heteroaryl moieties are optionally substituted with one to three substituents independently selected from R^{b}.

In other subclasses of these classes, R² is hydrogen, C₁₋₆ alkyl, cycloalkyl-C₀₋₆ alkyl, heterocycloalkyl-C₀₋₆ alkyl, or aryl-C₀₋₆ alkyl, wherein alkyl moieties are optionally substituted with one to three substituents independently selected from R^{a}, and cycloalkyl, heterocycloalkyl, aryl and heteroaryl moieties are optionally substituted with one to three substituents independently selected from R^{b}.

In yet other subclasses of these classes, R² is methyl, ethyl, n-propyl, isopropyl, t-butyl, n-butyl, cyclopropyl, cyclobutyl, cyclopentyl, cyclohexyl, or heterocycloalkyl-C₀₋₆ alkyl, wherein the heterocycloalkyl moiety is selected from azetidinyl, pyrrolidinyl, and pyridyl and phenyl-C₀₋₆ alkyl, wherein alkyl moieties are optionally substituted with one to three substituents independently selected from R^{a}, and cycloalkyl, heterocycloalkyl, and aryl moieties above are optionally substituted with one to three substituents independently selected from R^{b}.

In one particular embodiment of the invention, when R¹ is hydrogen or 2-hydroxyethyl, R² is other than 4-methansulfonamidophenethyl.

In other embodiments, R¹ and R² together with the nitrogen atom to which they are attached, form a 4- to 11-membered bridged or unbridged or spirocyclic heterocyclic ring, optionally containing one or two additional heteroatoms selected from N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom. In one class of this embodiment of the invention, R¹ and R² together with the nitrogen atom to which they are attached, form a 4- to 11-membered bridged or unbridged or spirocyclic heterocyclic ring, optionally containing one additional heteroatom selected from N, S, and O, optionally having one or more degrees of unsaturation, optionally fused to a 6-membered heteroaromatic or aromatic ring, either unsubstituted or substituted with an R^{b} substituent. In some subclasses of these classes, R¹ and R² together with the nitrogen atom to which they are attached, form a 4- to 11-membered bridged or unbridged or spirocyclic heterocyclic ring, optionally containing one additional heteroatom selected from N, S, and O, either unsubstituted or substituted with an R^{b} substituent. In other subclasses of these classes, R¹ and R² together with the nitrogen atom to which they are attached, form a 4- to 11-membered bridged or unbridged or spirocyclic heterocyclic ring selected from the group consisting of azetidinyl, pyrrolidinyl, piperidinyl, morpholinyl, 1-thia-4-azacyclohexyl, 2,5-diazabicyclo[2.2.2]octanyl, azacycloheptyl, 2-oxa-5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, 2,7-diazaspiro[4.4]nonyl, 2,7-diazaspiro[4.5]decyl, 2,7-diazaspiro[4.6]undecyl, 1,7-diazaspiro[4.4]nonyl, 2,6-dizaospiro[4.5]decyl, and 2,6-diazaspiro[4.6]undecyl, each of which is optionally substituted with an R^{b} substituent.

In yet other embodiments of the invention, R³ is hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁷R⁷, —CO₂R⁷, cyano, or —C(O)NR⁷R⁷; wherein alkyl, alkenyl and alkynyl, moieties are optionally substituted with one to four substituents independently selected from R^{a}, and cycloalkyl, heterocycloalkyl, aryl and heteroaryl moieties are optionally substituted with one to four substituents independently selected from R^{b}; and wherein sulfur-containing heterocyclic rings may be mono- or di-oxidized on the sulfur atom.

In some classes of these embodiments, R³ is hydrogen, halogen, C₁₋₈ alkyl, trifluoromethyl, C₂₋₆ alkenyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁷R⁷, —CO₂R⁷, or —C(O)NR⁷R⁷; wherein alkyl and alkenyl moieties are optionally substituted with one to three substituents independently selected from R^{a}, and cycloalkyl, heterocycloalkyl, aryl and heteroaryl moieties are optionally substituted with an R^{b} substituent.

In some subclasses of these classes, R³ is hydrogen, halogen, C₁₋₈ alkyl, trifluoromethyl, —OH, —OCH₃, —NH₂, —CO₂R⁷, or —C(O)NH₂; wherein alkyl moiety is optionally substituted with one to two substituents independently selected from R^{a}.

In other subclasses of these classes, R³ is hydrogen, halogen, C₁₋₈ alkyl, or trifluoromethyl, wherein alkyl moiety is optionally substituted with one to two substituents independently selected from R^{a}.

In yet other subclasses of these classes, R³ is hydrogen, halogen, methyl, ethyl, propyl, or trifluoromethyl, wherein alkyl moieties are optionally substituted with one to two substituents independently selected from R^{a}.

In still other embodiments, R⁴ is hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁷R⁷, —CO₂R⁷, or —C(O)NR⁷R⁷; wherein alkyl, alkenyl and alkynyl, moieties are optionally substituted with one to four substituents independently selected from R^{a}, and cycloalkyl, heterocycloalkyl, aryl and heteroaryl moieties are optionally substituted with one to four substituents independently selected from R^{b}; and wherein sulfur-containing heterocyclic rings may be mono- or di-oxidized on the sulfur atom.

In some classes within these embodiments, R⁴ is hydrogen, halogen, C₁₋₈ alkyl, trifluoromethyl, C₂₋₆ alkenyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁷R⁷, —CO₂R⁷, or —C(O)NR⁷; wherein alkyl and alkenyl moieties are optionally substituted with one to three substituents independently selected from R^{a}, and cycloalkyl, heterocycloalkyl, aryl and heteroaryl moieties are optionally substituted with an R^{b} substituent.

In some subclasses of these classes, R⁴ is hydrogen, halogen, C₁₋₈ alkyl, trifluoromethyl, cycloalkyl, cycloheteroalkyl, aryl-C₀₋₆ alkyl, heteroaryl, —OH, —OCH₃, —NH₂, —CO₂R⁷, or —C(O)NH₂; wherein alkyl moieties are optionally substituted with one to four substituents independently selected from R^{a}, and cycloalkyl, heterocycloalkyl, aryl and heteroaryl moieties are optionally substituted with an R^{b} substituent.

In other subclasses of these classes, R⁴ is hydrogen, C₁₋₈ alkyl, trifluoromethyl, cycloalkyl, cycloheteroalkyl, aryl, heteroaryl, —NH₂, —CO₂H, —CO₂CH₃, or —CO₂CH₂CH₃; wherein alkyl moieties are optionally substituted with one to two substituents independently selected from R^{a}, and cycloalkyl, heterocycloalkyl, aryl and heteroaryl moieties are optionally substituted with an R^{b} substituent.

In yet other subclasses of these classes, R⁴ is hydrogen, methyl, ethyl, propyl, trifluoromethyl, —CO₂H, —CO₂CH₃, or —CO₂CH₂CH₃; wherein alkyl moieties are optionally substituted with one to three substituents independently selected from R^{a}.

In still other subclasses of these classes, R⁴ is hydrogen, methyl, ethyl, —CO₂H, or —CO₂CH₃.

In other embodiments of the invention, R³ and R⁴ together with the ring carbon atoms to which they are attached form a 5- to 7-membered heterocycloalkyl or cycloalkyl ring, optionally substituted with one to four substituents independently selected from R^{b}. In some classes of these embodiments, R³ and R⁴ together with the ring carbon atoms to which they are attached form a 5- to 7-membered heterocycloalkyl or cycloalkyl ring, optionally substituted with an R^{b} substituent. In some subclasses of these classes, R³ and R⁴ together with the ring carbon atoms to which they are attached form cyclohexyl ring, optionally substituted with an R^{b} substituent. In other subclasses of these classes, R³ and R⁴ together with the ring carbon atoms to which they are attached form a cyclohexyl ring, optionally substituted with oxo or hydroxy.

In some embodiments of the invention, R⁵ is hydrogen, halogen, C₁₋₆ alkyl, perfluoro C₁₋₆ alkyl, —OR⁷, or —NR⁷R⁷.

In some classes of these embodiments, R⁵ is hydrogen, halogen, methyl, trifluoromethyl, hydroxy, methoxy, phenoxy, —NH₂, —NH(CH₃), or —N(CH₃)₂.

In yet other classes of these embodiments, R⁵ is hydrogen, halogen, methyl, trifluoromethyl, hydroxy, methoxy, phenoxy, —NH₂, —NH(CH₃), or —N(CH₃)₂.

In some subclasses of these classes, R⁵ is hydrogen, halogen, methyl, trifluoromethyl, hydroxy, or methoxy.

In other subclasses of these classes, R⁵ is hydrogen.

In other embodiments of the invention, R⁶ is —CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ, —(CH₂)_{n CON(R}⁷)₂,—CH₂)ₙCO₂R⁷, —(CH₂)ₙCOR⁷, —(CH₂)ₙNR⁷C(O)R⁷, —(CH₂)ₙNR⁷C(O)(CH₂)ₙSR⁷, —(CH₂)ₙCO₂R⁷, —(CH₂)ₙCOR⁷, —(CH₂)ₙNR⁷C(O)R⁷, —(CH₂)ₙNR⁷C(O)(CH₂)ₙSR⁷, —(CH₂)ₙSO₂N(R⁷)₂, —(CH₂)ₙOR⁷, —(CH₂)ₙOC(O)R⁷, —(CH₂)ₙOC(O)OR⁷, —(CH₂)ₙOC(O)N(R⁷)₂, —(CH₂)ₙN(R⁷)₂, or —(CH₂)ₙNR⁷SO₂N(R⁷)₂, wherein one or two of the hydrogen atoms in (CH₂)ₙ may be substituted with R^{a}.

In some classes of these embodiments of the invention, R⁶ is —(CH₂)ₙ—R⁷, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —CH₂)ₙCON(R⁷)₂, —(CH₂)ₙNR⁷C(O)R⁷, —(CH₂)ₙNR⁷C(O)(CH₂)ₙSR⁷, —(CH₂)ₙNR⁷C(O)N(R⁷)₂, —(CH₂)ₙNHSO₂R⁷, —(CH₂)ₙN(R⁷)₂, or —(CH₂)ₙNR⁷SO₂N(R⁷)₂, wherein one or two of the hydrogen atoms in (CH₂)ₙ may be substituted with R^{a}.

In some subclasses of these classes, R⁶ is —R⁷, -heteroaryl-R⁷, —CON(R⁷)(CH₃), —CH₂CONHR⁷, —CH₂CON(R⁷)(CH₃), —CH₂NHC(O)R⁷, —NHC(O)R⁷, —(CH₂)₂ₙNHC(O)(CH₂)ₙSR⁷, —(CH₂)ₙNHC(O)N(CH₃)(R⁷), —(CH₂)ₙNHC(O)NH(R⁷), —(CH₂)ₙNHSO₂R⁷, —NH(R⁷), —N(COCH₃)(R⁷), —(CH₂)ₙNH(R⁷), or —(CH₂)ₙN(COCH₃)(R⁷), wherein one or two of the hydrogen atoms in (CH₂)ₙ may be substituted with R^{a}.

In yet other embodiments of the invention, each R⁷ is independently hydrogen, C₁₋₆ alkyl, aryl C₀₋₃ alkyl, heteroaryl C₀₋₃ alkyl, cycloalkyl C₀₋₃ alkyl, heterocycloalkyl C₀₋₃ alkyl, aryl C₂₋₃ alkenyl, heteroaryl C₂₋₃ alkenyl, cycloalkyl C₂₋₃ alkenyl, or heterocycloalkyl C₂₋₃ alkenyl, wherein the alkyl and alkenyl moieties are optionally substituted with one to four substituents selected from R^{a}, and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom.

In some classes of theses embodiments of the invention, the alkyl and alkenyl moieties in R⁷ are optionally substituted with one to three substituents selected from R^{a}, and wherein the aryl, heteroaryl, cycloalkyl and heterocycloalkyl moieties in R⁷ are optionally independently substituted with one to three substituents selected from R^{b}; and wherein sulfur-containing heterocyclic rings may be mono- or di-oxidized on the sulfur atom.

In another class of this embodiment of the invention, each R⁷ is independently hydrogen, C₁₋₆ alkyl, aryl (wherein aryl is selected from the group consisting of phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl), heteroaryl (wherein heteroaryl is selected from the group consisting of pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl), cycloalkyl (wherein cycloalkyl is selected from the group consisting of cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl), heterocycloalkyl (wherein heterocycloalkyl is selected from the group consisting of azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl), aryl C₁₋₃ alkyl (wherein the aryl moiety is selected from the group consisting of phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl) piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl), heteroaryl C₁₋₃ alkyl (wherein the heteroaryl moiety is selected from the group consisting of pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl), cycloalkyl C₁₋₃ alkyl (wherein the cycloalkyl moiety is selected from the group consisting of cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl), heterocycloalkyl C₁₋₃ alkyl (wherein the heterocycloalkyl moiety is selected from the group consisting of azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl), aryl C₂₋₃ alkenyl (wherein the aryl moiety is selected from the group consisting of phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl), heteroaryl C₂₋₃ alkenyl (wherein the heteroaryl moiety is selected from the group consisting of pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl), cycloalkyl C₂₋₃ alkenyl (wherein the cycloalkyl moiety is selected from the group consisting of cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl), and heterocycloalkyl C₂₋₃ alkenyl (wherein the heterocycloalkyl moiety is selected from the group consisting of azetidinyl, pyridyl, pyrrolidinyl, piperidinyl, piperazinyl, imidazolidinyl, morpholinyl, ₁-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl); wherein the alkyl moieties are optionally substituted with one to three substituents selected from R^{a}, and wherein the aryl, heteroaryl, cycloalkyl and heterocycloalkyl moieties are independently substituted with one to three substituents selected from R^{b} and wherein sulfur-containing heterocyclic rings may be mono- or di-oxidized on the sulfur atom.

In other embodiments of the invention, each R^{a} is independently selected from the group consisting of —OR^{d}, —NR^{d}S(O)ₘR^{d}, —NO₂, halogen, —S(O)ₘR^{d}, —SR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d})₂, —OC(O)R^{d}, —CN, —C(O)N(R^{d})₂, 13 NR^{d}C(O)R^{d}, —OC(O)N(R^{d})₂, —NR^{d}C(O)OR^{d}, —NR^{d}C(O)N(R^{d})₂, —CR^{d}(N—OR^{d}), —CF₃, cycloalkyl, cycloheteroalkyl, and oxo.

In some classes of these embodiments, each R^{a} is independently selected from the group consisting of —OR^{d}, —NHSO₂CH₃, —NO₂, halogen, —S(O)ₘCH₃, —SCH₃, —SCF₃, —S(O)₂OR^{d}, —S(O)ₚN(R^{d})₂, —N(CH₃)₂, —NH₂, —O(CR^{d}R^{d})ₙN(R^{d})₂, —C(O)R^{d}, —CO₂H, —CO₂CH₃, t-butyloxycarbonyl, —CO₂(CR^{d}R^{d})ₙCON(R^{d})₂, —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R^{d})₂, —CR^{d}(N—OR^{d}), —CF₃, cycloalkyl, cycloheteroalkyl, and oxo.

In other embodiments of the invention, each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl are optionally substituted with one to four R^{c} substituents.

In some classes of these embodiments, each R^{b} is independently selected from the group consisting of R^{a}, —Sn(CH₃)₃, C₁₋₁₀ alkyl, C₂₋₁₀ alkenyl, heteroaryl, and aryl-C₀₋₁₀ alkyl; wherein alkyl, alkenyl, cycloalkyl, cycloheteroalkyl, heteroaryl, and aryl are optionally substituted with one to four substituents selected from a group independently selected from R^{c}.

In some subclasses of these classes, each R^{b} is independently selected from the group consisting of R^{a}, —Sn(CH₃)₃, C₁₋₆ alkyl, C₂₋₆ alkenyl, heteroaryl, and aryl-C₀₋₁₀ alkyl; wherein alkyl, alkenyl, cycloalkyl, cycloheteroalkyl, heteroaryl, and aryl moieties in R^{a} and R^{b} are optionally substituted with one to four substituents independently selected from R^{c}.

In yet other embodiments of the invention, each R^{c} is independently selected from the group consisting of halogen, amino, carboxy, C₁₋₄ alkyl, C₁₋₄ alkoxy, aryl C₀₋₄ alkyl, hydroxy, —CF₃, —OC(O)C₁₋₄ alkyl, —OC(O)N(R^{d})₂, and (12) aryloxy.

In still other embodiments of the invention, each R^{d} is independently selected from the group consisting of hydrogen, C₁₋₆ alkyl, C₂₋₆ alkenyl; C₂₋₆ alkynyl; cycloalkyl; cycloalkyl-C₁₋₆ alkyl; cycloheteroalkyl-C₀₋₆ alkyl; aryl-C₀₋₆ alkyl; and heteroaryl-C₀₋₆ alkyl; wherein the alkyl, alkenyl, alkynyl, cycloalkyl, cycloheteroalkyl, heteroaryl, and aryl in R^{d} are optionally substituted with one to four substituents independently selected from R^{e}. In some classes of these embodiments of the invention, the alkyl, alkenyl, alkynyl, cycloalkyl, cycloheteroalkyl, heteroaryl, and aryl in R^{d} are optionally substituted with one to two substituents independently selected from R^{e}.

In other embodiments of the invention, each R^{e} is selected from the group consisting of halo, methyl, methoxy, trifluoromethyl, trifluoromethoxy, and hydroxy.

In still other embodiments of the invention, each m is independently selected from 1 and 2. In some classes of these embodiments, m is 1. In other classes of these embodiments m is 2.

In yet other embodiments of the invention, each n is independently elected from 0, 1, 2, 3, 4, and 5. In some classes of these embodiments, each n is independently selected from 0, 1, 2, 3, and 4. In some subclasses of these classes, n is selected from 0, 1, 2, and 3. In other subclasses of these classes, n is selected from 0, 1, and 2. In still other subclasses of these classes, n is 0.

In still other embodiments of the invention, each p is independently selected from the group consisting of 0, 1, and 2. In some classes of these embodiments, p is 0. In other classes of these embodiments, p is 1. In still other classes of these embodiments, p is 2.

Yet in other embodiments, a is 0, 1, or 2. In some classes within these embodiments, a is 0 or 1. Still in other classes within these embodiments, a is 0.

In some embodiments, Y is —NR¹R².

In other embodiments, R¹ and R² are hydrogen.

Still in other embodiments, R³ and R⁴ are hydrogen.

Yet in other embodiments, R⁶ is hydrogen.

In other embodiments, a is 0.

Still yet in other embodiments, R⁴ is —OH.

Still further, combinations of the various groups in particular embodiments described herein form other embodiments. For example, in one particularly embodiment Y is —NR¹R², R¹ and R² are hydrogen, R³ and R⁴ are hydrogen, R⁶ is hydrogen, and a is 0. In another particular embodiments, R⁴ is —OH, R⁶ is hydrogen, and a is 0. In this manner, a variety of compounds are embodied within the invention.

Another aspect of the invention provides a composition comprising a therapeutically effective amount of a Compound of Formula I and a pharmaceutically acceptable carrier.

Yet another aspect of the invention provides a method for treating bacterial infection in a subject comprising administering to the subject a Compound of Formula I. In some embodiments, the bacterial infection comprises lung infection, wound infection, urinary tract infection or a combination thereof. Within these embodiments, in some instances the bacterial infection is in the lung of cystic fibrosis patient.

Still in other embodiments, the bacterial infection is caused by a gram negative bacteria.

In other embodiments, the bacterial infection is caused by bacteria comprising Burkholderia cepacia, Pseudomonas aeruginosa, Stenotrophomonas maltophilia, A. xylosoxidans, A. baumannii, or a combination thereof.

Another particular aspect of the invention provides a method for treating bacterial infection caused by a bacteria comprising Burkholderia cepacia, Pseudomonas aeruginosa, and Stenotrophomonas maltophilia, A. xylosoxidans, A. baumannii, or a combination thereof in a subject by, said method comprising administering to the subject a Compound of Formula I.

In some embodiments, the bacteria infection comprises lung infection, wound infection, urinary tract infection or a combination thereof.

In other embodiments, the bacterial infection comprises lung infection of a cystic fibrosis patient.

Another aspect of the invention provides a method for treating bacterial infection comprising lung infection, wound infection, urinary tract infection, or a combination thereof in a subject. The method generally comprises administering to the subject a Compound of Formula I.

In some embodiments, the subject has cystic fibrosis.

Still in other embodiments, the bacterial infection is caused by a bacteria comprising Burkholderia cepacia, Pseudomonas aeruginosa, and Stenotrophomonas maltophilia, A. xylosoxidans, A. baumannii, or a combination thereof.

Yet another aspect of the invention provides a method for treating fungal infection in a subject comprising administering to the subject a Compound Formula I.

In some embodiments, the fungal infection is caused by a fungus comprising Cryptococcus neoformans, Candida albicans, Aspergillus fumigatus, Trichophyton mentagrophytes, or a combination thereof. Within these embodiments, in some instances the subject has immunodeficiency. In some cases within these instances, the subject has AIDS.

### Synthesis

Compounds of the invention are commercially available or can be made by a variety of methods known to one skilled in the art. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. Some of the particular compounds can be made followings the procedures disclosed in U.S. Pat. No. 7,084,156, which is incorporated herein by reference in its entirety. Other compounds of the invention can be made by various modifications suggested to one skilled in the art having referred to the disclosure contained in this Application.

The starting materials and the intermediates can be isolated and purified if desired using conventional techniques, including but not limited to, filtration, distillation, crystallization, chromatography, and the like. Such materials can be characterized using conventional means, including physical constants and spectral data.

Typically, the reactions are conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

### Utility

The compounds of the invention have antimicrobial activity, and therefore are useful in the treatment of various microbial infections such as bacterial infection and fungal infection. In some embodiments, the bacterial infection comprises lung infection, wound infection, urinary tract infection or a combination thereof. Within these embodiments, in some instances the bacterial infection is in the lung of cystic fibrosis patient.

In some embodiments, the bacterial infection is caused by a gram negative bacteria such as Burkholderia cepacia, Pseudomonas aeruginosa, Stenotrophomonas maltophilia, A. xylosoxidans, A. baumannii, or a combination thereof.

Compounds of the invention can also be used to treat fungal infections such as those caused by a fungus comprising Cryptococcus neoformans, Candida albicans, Aspergillus fumigatus, Trichophyton mentagrophytes, or a combination thereof. In some instances compounds of the invention are useful in treating fungal infection in a subject having immunodeficiency such as those caused by AIDS.

### Bacterial Infection in CF Patients

Several bacterial species are responsible for chronic infections and early mortality in cystic fibrosis (CF) patients. Some of these species are quite resistant to a variety of antibiotics, so treatment options have become more limited in patients colonized with these bacterial species. Despite the need for new antibiotics, there are few new antimicrobials in the pipelines of pharmaceutical companies. Compounds of the invention are active against a number of bacterial species, including Burkholderia cepacia, Pseudomonas aeruginosa, and Stenotrophomonas maltophilia, which are common causes of lung infection in CF patients.

Respiratory failure as the result of chronic respiratory tract infections is the most significant cause of both morbidity and mortality in CF patients. Infants with CF have normal lungs free of bacteria. Typically, it takes several months or even years for chronic infections to develop in the lungs of CF children. Several bacterial species are primary culprits responsible for the premature mortality associated with CF, including Pseudomonas aeruginosa, Staphylococcus aureus, and the Burkholderia cepacia complex. Secondary pathogens of the respiratory tracts of CF patients include some additional bacterial species (Haemophilus influenzae, Mycobacterium sp., Stenotrophomonas maltophilia, and Alcaligenes xylosoxidans), fungi (Aspergillus fumigatus), and viruses (respiratory syncytical and influenza virus).

Of the main bacterial species colonizing lungs of CF patients, P. aeruginosa is found in the lungs of at least 60% of all patients suffering from the disease. Without being bound by any theory, it is believed that the emergence of mucoid varieties of this bacterium signals the onset of chronic infections and herald's significant lung damage and regular hospitalization. Recently, an epidemic strain with greater virulence and higher antibacterial resistance has emerged, causing more serious lung infections. It is believed that most of the infections are caused by clonal strains and are acquired independently. Loss-of-function mutations frequently arise in these bacteria, some that bring on greater antibiotic resistance while others result in greater adaption to living in the airways of CF patients.

Staphylococcus aureus is also found frequently in the lungs of CF patients, particularly among children. It is one of the first species to colonize the CF respiratory tract. Approximately 6% of S. aureus isolates found in CF patients are resistant to methicillin.

While the B. cepacia complex infects just 3% of American CF patients, those patients that are infected with this organism have a poorer quality of life than other CF patients. In addition, the patients who are infected with B. cepacia are segregated from other CF patients, which further erode the quality of life in these patients. A fifth of the patients infected with the B. cepacia complex suffers from a condition known as “cepacia syndrome” , resulting in a dramatic drop in lung function, many bouts of bacteremia, and a higher incidence of lung failure. More importantly, some strains of the B. cepacia complex have been isolated that are resistant to all conventional antibiotics. Currently, resistance to drugs like piperacillin, ceftazidime, and pipericillin-tazobactam occurs in a third or more of B. cepacia isolates tested for antibiotic resistance. Several outbreaks of B. cepacia complex have been noted in CF care centers that have been tied to unique strains that wreak havoc on a patient's health. Isolation practices have been implemented with some success, but these practices do not eradicate the bacteria that have initiated the infections.

The secondary bacterial pathogen S. maltophilia is on the rise in CF adults. During aerosolized tobramycin treatment for patients afflicted with P. aeruginosa lung infections, 19% of the population acquired S. maltophilia. One study showed that patients who were colonized with S. maltophilia had higher morbidity and mortality compared to a population not infected with S. maltophilia.

### Testing

The pharmacology of the compounds of this invention was determined by art recognized procedures. The in vitro techniques for determining antimicrobial activity of Compounds of Formula I are described in the Examples section below.

### Administration and Pharmaceutical Composition

The invention includes pharmaceutical compositions comprising at least one Compound of Formula I, or an individual tautomer, a mixture of tautomers, an individual isomer, racemic or non-racemic mixture of isomers, or a pharmaceutically acceptable salt or solvate thereof, together with at least one pharmaceutically acceptable carrier, and optionally other therapeutic and/or prophylactic ingredients.

In general, the compounds of the invention is administered in a therapeutically effective amount by any of the accepted modes of administration for agents that serve similar utilities. Suitable dosage ranges are typically 1 to 500 mg daily, often 1 to 100 mg daily, and more often 1 to 30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the invention for a given microbial infection.

In general, compounds of the invention is administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. Typically, the manner of administration is oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention can be formulated in a wide variety of oral administration dosage forms. The pharmaceutical compositions and dosage forms can comprise a compound or compounds of the present invention or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid that is a mixture with the finely divided active component. In tablets, the active component generally is mixed with the carrier having the necessary binding capacity in suitable proportions and compacted in the shape and size desired. The powders and tablets typcially contain from about one (1) to about seventy (70) percent of the active compound. Suitable carriers include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention can also be formulated for parenteral administration (e.g., by injection, for example bolus injection or continuous infusion) and may be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

The compounds of the invention also can be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the invention can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention can be formulated for vaginal administration. Pessaries, tampons, creams, gels, pastes, foams or sprays containing in addition to the active ingredient such carriers as are known in the art to be appropriate.

The compounds of the invention can also be formulated for nasal administration. The solutions or suspensions are applied directly to the nasal cavity by conventional means, for example, with a dropper, pipette or spray. The formulations may be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compounds of the invention also can be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy, 1995, edited by E. W. Martin, Mack Publishing Company, 19th edition, Easton, Pa.

Additional objects, advantages, and novel features of this invention will become apparent to those skilled in the art upon examination of the following examples thereof, which are not intended to be limiting.

### EXAMPLES

### Example 1

This Example illustrates a method for isolation and identification of 2-aminoquinoline from the fungus, Leucopaxillus albissimus.

The dried fungal fruiting bodies (mushrooms) of L. albissimus were collected primarily from Mendocino and Sonoma counties of California during the month of January and then air dried in closed paper bags to protect the fungal material from exposure to light. In an exemplary preparation, 10.31 g of this dried fungal material was ground in a blender and placed into a 250 mL Erlenmeyer flask with 5% methanol-95% dichloromethane. The flask was sealed with a rubber stopper and stirred over a stir plate for 24 h. The marc was removed by filtration using a Buchner funnel and extensive washing with dichloromethane. Removal of the volatiles and thorough drying under reduced pressure yielded the crude extract as a sticky brown gum weighing 0.51 mg (4.9%).

The crude extracts were typically fractionated by successive thin-layer chromatography using 500 mm thickness Whatman 20 cm×20 cm, 60Å silica gel plates with fluorescent indicator, eluting initially with 5% methanol-95% dichloromethane and then 50% isopropanol-50% acetonitrile. This bioassay-directed fractionation allowed for the identification of the major bioactive component, which inhibited the growth of several strains of bacteria in the Kirby-Bauer disc diffusion assay. The active component could be visualized under short wave UV light (254 nm) as a relatively low R_{f}, violet-colored band following development of the TLC plates. Final purification was accomplished by excising the active bands from the TLC plates, extracting into dichloromethane, evaporating, and then subjecting to high performance liquid chromatography (HPLC). HPLC purification employed a Waters HPLC system with diode array detector, a preparative scale, reverse phase C-18 column, and a 1% ammonia-45% methanol-44% water solution as the eluant. The major component that eluted from the column retained the previously observed bioactivity, and this purified sample was also used for subsequent structure elucidation studies.

The major active component from L. albissimus was determined to be 2-aminoquinoline (2-AQ), also known as 2-quinolylamine, on the basis of the spectral data acquired for the HPLC-purified sample. Thus, its positive ionization high-resolution mass spectrum (HRMS) showed an exact mass of 145.07573 g/mol for the M+1 ion, indicating a molecular formula of C₉H₈N₂ consistent with that of 2-AQ. The proton and carbon NMR spectra were essentially identical to those reported in the literature for 2-AQ (Pfister, J. R., J. Nat. Prod., 1988, 51(5), 969-970). The IR spectrum (thin film method) was superimposable on the IR spectrum for 2-AQ contained in the online SDBS spectral database. Finally, the HPLC retention time and NMR spectra of the purified natural product were compared with those of an authentic synthetic sample obtained through Sigma-Aldrich, and these were identical, as were the biological activities of the two samples.

### Example 2

A compound of the invention, 2-aminoquinoline, was tested for activity against gram-negative bacteria. Some of the results are shown in Table 1 below.

TABLE 1
MIC of purified 2-aminoquinoline against Gram-negative
bacterial species.

| Bacterial strains | MIC (μg/mL) |
|---|---|
| Burkholderia cepacia | 40 |
| Burkholderia multivorans AU 5573 | 90 |
| Burkholderia multivorans AU 7455 | 48 |
| Burkholderia multivorans AU 10398 | 16 |
| Burkholderia cenocepacia AU 6550 | 26 |
| Burkholderia cenocepacia AU 9292 | 58 |
| Burkholderia cenocepacia AU 10321 | 40 |
| Acinetobacter baumannii | 128 |
| Achromobacter xylosoxidans | 64 |
| Stenotrophomonas maltophilia | 40 |
| Pseudomonas aeruginosa | 133 |

### Example 3

Some of the compounds of the invention were tested against Burkholderia cepacia. Mininum inhibitory concentration (MIC) are shown in Table 2 below.

TABLE 2
MIC of some of the compounds of the invention
against Burkholderia cepacia.

| Compound | MIC (μg/mL) |
|---|---|
| 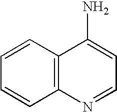 | 40 |
| 4-aminoquinoline | |
| 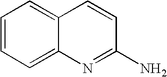 | 32 |
| 2-aminoquinoline | |
| 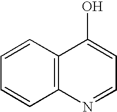 | >128 |
| 4-hydroxyquinolone | |
| 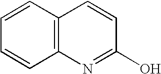 | >128 |
| 2-hydroxyquinolone | |

### Example 4

Table 3 below shows some of the therapeutic uses for various bacterial infections of a compound of the invention including 2-aminoquinoline.

TABLE 3
Exemplary therapeutic uses of 2-aminoquinoline.

| Infection | Bacterial Species |
|---|---|
| CF Patient Lung Infections | A. xylosoxidans, B. cepacia complex, |
| | P. aeruginosa, S. maltophilia |
| Wound Infections | A. baumannii, A. xylosoxidans, B. cepacia |
| | complex, P. aeruginosa, S. maltophilia |
| Urinary Tract Infections | A. baumannii, A. xylosoxidans, B. cepacia |
| | complex, P. aeruginosa, S. maltophilia |

### Example 5

Table 4 below shows antifungal activity of 2-aminoquinoline.

TABLE 4
Minimum inhibitory concentrations of 2-aminoquinoline
against fungal species.

| Fungal Species | MIC (μg/mL) |
|---|---|
| Candida albicans | 320 |
| Cryptococcus neoformans | 160 |
| Aspergillus fumigatus | 160 |
| Trichophyton mentagrophytes | 160 |

### Example 6

This example illustrates methods for testing cytotoxicity of a compound of the invention.

The cytotoxicity testing of 2-aminoquinoline (2-AQ) was done according to Hathaway et al. (“The acridine orange viability test applied to bone marrow cells. I. Correlation with trypan blue and eosin dye exclusion and tissue culture transformation”, Blood, 1964, 23, 517-525) and Newberry and Sanford (“Defective cellular immunity in renal failure: depression of reactivity of lymphocytes to phytohemagglutinin by renal failure serum,” J. Clin. Invest., 1971, 50, 1261-1271). Briefly, J774A.1 (murine) and U937 (human) monocytic cells were mixed with different concentrations of 2-AQ overnight. The next day the wells were washed with PBS and trypan blue was added (0.5%) for 15 min. The number of injured/dead (blue) versus uninjured (clear) cells were tabulated.

The results showed that about 1.5 mg/ml of 2-AQ was needed to injure 50% of both J774A.1 and U937 cells, whereas a concentration of 250-500 μg/mL caused some minor injury. Concentrations below 250 μg/mL had no significant effect on both cell lines.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A method for treating bacterial infection in a subject comprising administering to the subject in need of such treatment a compound of the formula: 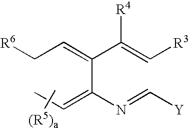
or an isomer, tautomer, prodrug or a pharmaceutically acceptable salt thereof,
wherein
Y is —NR¹R², —OR⁷, or —SR⁷;
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷;
R⁶ is selected from the group consisting of hydrogen, halogen, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ—NR⁷CO₂R⁷, —(CH₂)ₙ—NR⁷C(O)N(R⁷)₂, —(CH₂)ₙ—NR⁷SO₂R⁷, —(CH₂)ₙ—S(O)₂R⁷, —(CH₂)ₙ—SO₂N(R⁷)₂, —(CH₂)ₙ—OR⁷, —(CH₂)ₙ—OC(O)R⁷—(CH₂)ₙ—OC(O)OR⁷, —(CH₂)ₙ—OC(O)N(R⁷)₂, —(CH₂)ₙ—N(R⁷)₂, and —(CH₂)ₙ—NR⁷SO₂N(R⁷)₂, wherein one or two of the hydrogen atoms in (CH₂)ₙ, may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R^{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C₀₋₄ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₂₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

2. The method of claim 1, wherein the bacterial infection comprises lung infection, wound infection, urinary tract infection or a combination thereof.

3. The method of claim 2, wherein the bacterial infection is in the lung of cystic fibrosis patient.

4. The method of claim 1, wherein the bacterial infection is caused by a gram negative bacteria.

5. The method of claim 1, wherein the bacterial infection is caused by bacteria comprising Burkholderia cepacia, Pseudomonas aeruginosa, Stenotrophomonas maltophilia, A. xylosoxidans, A. baumannii, or a combination thereof.

6. The method of claim 1, wherein Y is —NR¹R².

7. The method of claim 6, wherein R¹ and R² are hydrogen.

8. The method of claim 1, wherein R³ and R⁴ are hydrogen.

9. The method of claim 1, wherein R⁶ is hydrogen.

10. The method of claim 1, wherein a is 0.

11. The method of claim 1, wherein R⁴ is —OH.

12. A method for treating bacterial infection caused by a bacteria comprising Burkholderia cepacia, Pseudomonas aeruginosa, and Stenotrophomonas maltophilia, A. xylosoxidans, A. baumannii, or a combination thereof in a subject, said method comprising administering to the subject in need of such treatment a compound of the formula: 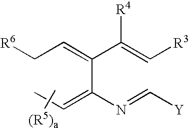
or an isomer, tautomer, prodrug or a pharmaceutically acceptable salt thereof,
wherein
Y is —NR¹R², —OR⁷, or —SR⁷;
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷; —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ—NR⁷CO₂R⁷, —(CH₂)ₙ—NR⁷C(O)N(R⁷)₂, —(CH₂)ₙ—NR⁷SO₂R⁷, —(CH₂)ₙ—S(O)₂R⁷, —(CH₂)ₙ—SO₂N(R⁷)₂, —(CH₂)ₙ—OR⁷, —(CH₂)ₙ—OC(O)R⁷, —(CH₂)ₙ—OC(O)OR⁷, —(CH₂)ₙ—OC(O)N(R⁷)₂, —(CH₂)ₙ—N(R⁷)₂, and —(CH₂)ₙ—NR⁷SO₂N(R⁷)₂, wherein one or two of the hydrogen atoms in (CH₂)ₙ may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R^{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C₀₋₄ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₀₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

13. The method of claim 12, wherein the bacteria infection comprises lung infection, wound infection, urinary tract infection or a combination thereof.

14. The method of claim 12, wherein Y is —NR¹R².

15. The method of claim 14, wherein R¹ and R² are hydrogen.

16. The method of claim 12, wherein R³ and R⁴ are hydrogen.

17. The method of claim 12, wherein R⁶ is hydrogen.

18. The method of claim 12, wherein a is 0.

19. The method of claim 12, wherein R⁴ is —OH.

20. The method of claim 13, wherein the bacterial infection comprises lung infection of a cystic fibrosis patient.

21. A method for treating bacterial infection comprising lung infection, wound infection, urinary tract infection, or a combination thereof in a subject, said method comprising administering to the subject in need of such treatment a compound of the formula: 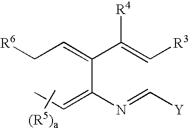
or an isomer, tautomer, prodrug or a pharmaceutically acceptable salt thereof,
wherein
Y is —NR¹R², —OR⁷, or —SR⁷;
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷;
R⁶ is selected from the group consisting of hydrogen, halogen, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ-NR⁷CO₂R⁷, —(CH₂)ₙ—NR⁷C(O)N(R⁷)₂, —(CH₂)ₙ—NR⁷SO₂R⁷, —(CH₂)ₙ—S(O)₂R⁷, —(CH₂)ₙ—SO₂N(R⁷)₂, —(CH₂)ₙ—OR⁷, —(CH₂)ₙ—OC(O)R⁷, —(CH₂)ₙ—OC(O)OR⁷, —(CH₂)ₙ—OC(O)N(R⁷)₂, —(CH₂)ₙ—N(R⁷)₂, and —(CH₂)ₙ—NR⁷SO₂N(R⁷)₂, wherein one or two of the hydrogen atoms in (CH₂)ₙ may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}, —S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R^{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C₀₋₄ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₀₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

22. The method of claim 21, wherein the subject has cystic fibrosis.

23. The method of claim 21, wherein the bacterial infection is caused by a bacteria comprising Burkholderia cepacia, Pseudomonas aeruginosa, and Stenotrophomonas maltophilia, A. xylosoxidans, A. baumannii, or a combination thereof.

24. The method of claim 23, wherein Y is —NR¹R².

25. The method of claim 24, wherein R¹ and R² are hydrogen.

26. The method of claim 25, wherein R³ and R⁴ are hydrogen.

27. The method of claim 26, wherein R⁶ is hydrogen.

28. The method of claim 27, wherein a is 0.

29. The method of claim 25, wherein R⁴ is —OH.

30. A method for treating fungal infection in a subject comprising administering to the subject in need of such treatment a compound of the formula: 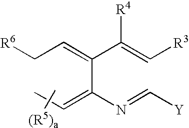
or an isomer, tautomer, prodrug or a pharmaceutically acceptable salt thereof,
wherein
Y is —NR¹R², —OR⁷, or —SR⁷;
each of R¹ and R² is independently selected from the group consisting of hydrogen; C₁₋₆ alkyl; C₂₋₆ alkenyl; C₂₋₆ alkynyl; cycloalkyl-C₀₋₆ alkyl; heterocycloalkyl-C₀₋₁₀ alkyl; aralkyl; and heteroaralkyl; wherein alkyl, alkenyl, and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 11-membered bridged or unbridged or spirocyclic heterocyclic ring, optionally containing one or two additional heteroatoms selected from the group consisting of N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
each of R³ and R⁴ is independently selected from the group consisting of hydrogen, halogen, C₁₋₈ alkyl, perfluoro C₁₋₆ alkyl, C₂₋₆ alkenyl, C₂₋₆ alkynyl, cycloalkyl-C₀₋₆ alkyl, cycloheteroalkyl-C₀₋₆ alkyl, aryl-C₀₋₆ alkyl, heteroaryl-C₀₋₆ alkyl, —OR⁷, —NR⁸R⁹, —CO₂R⁷, cyano, and —C(O)NR⁸R⁹; wherein alkyl, alkenyl and alkynyl, moieties of R³ and R⁴ are independently optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
each of R⁵ is independently selected from the group consisting of halogen, C₁₋₆ alkyl, C₁₋₆ haloalkyl, —OR⁷, and —NR⁷R⁷;
R⁶ is selected from the group consisting of hydrogen, halogen, —(CH₂)ₙ—R⁷, —(CH₂)ₙ-aryl-R⁷, —(CH₂)ₙ-heteroaryl-R⁷, —(CH₂)ₙ-heterocycloalkyl-R⁷, —(CH₂)ₙ—CN, —(CH₂)ₙ—CON(R⁷)₂, —(CH₂)ₙ—CO₂R⁷, —(CH₂)ₙ—COR⁷, —(CH₂)ₙ—NR⁷C(O)R⁷, —(CH₂)ₙ—NR⁷C(O)—(CH₂)ₙ—SR⁷, —(CH₂)ₙ—NR⁷CO₂R⁷, —(CH₂)ₙ—NR⁷C(O)N(R⁷)₂, —(CH₂)ₙ—NR⁷SO₂R⁷, —(CH₂)ₙ—S(O)₂R⁷, —(CH₂)ₙ—SO₂N(R⁷)₂, —(CH₂)ₙ—OR⁷, —(CH₂)ₙ—OC(O)R⁷—(CH₂)ₙ—OC(O)OR⁷, —(CH₂)ₙ—OC(O)N(R⁷)₂, —(CH₂)ₙ—N(R⁷)₂, and —(CH₂)ₙ—NR⁷SO₂N(R⁷)₂, wherein one or two of the hydrogen atoms in (CH₂)ₙ may be substituted with R^{a};
R⁷ is independently selected at each occurrence from the group consisting of hydrogen, C₁₋₆ alkyl, aryl-C₀₋₃ alkyl, heteroaryl-C₀₋₃ alkyl, cycloalkyl-C₀₋₃ alkyl, heterocycloalkyl-C₀₋₃ alkyl, aryl-C₂₋₃ alkenyl, heteroaryl-C₂₋₃ alkenyl, cycloalkyl-C₂₋₃ alkenyl, and heterocycloalkyl-C₂₋₃ alkenyl, wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from the group consisting of —OR^{d}, —NR^{d}S(O)ₘR^{d}, —NO₂, halogen, —S(O)ₚR^{d}—S(O)₂OR^{d}, —S(O)ₚN(R^{d})₂, —N(R^{d})₂, —O(CR^{d}R^{d})ₙN(R^{d})₂, —C(O)R^{d}, —CO₂R^{d}, —CO₂(CR^{d}R^{d})ₙCON(R^{d}), —OC(O)R^{d}, —CN, —C(O)N(R^{d})₂, —NR^{d}C(O)R^{d}, —OC(O)N(R^{d})₂, —NR^{d}C(O)OR^{d}, —NR^{d}C(O)N(R^{d})₂, —CR^{d}(N—OR^{d})₂, —CF₃, cycloalkyl, cycloheteroalkyl, and oxo;
each R^{b} is independently selected from the group consisting of R^{a}, —Sn(CH₃)₃, C₁₋₁₀ alkyl, C₂₋₁₀ alkenyl, C₂₋₁₀ alkynyl, heteroaryl, and aryl-C₀₋₁₀ alkyl; wherein alkyl, alkenyl, alkynyl, cycloalkyl, cycloheteroalkyl, heteroaryl, and aryl in R^{b} are optionally substituted with one to four substituents selected from a group independently selected from R^{c};
each R^{c} is independently selected from halogen, amino, carboxy, C₁₋₄ alkyl, C₁₋₄ alkoxy, aryl-C₀₋₄ alkyl, hydroxy, —CF₃, —OC(O)—C₁₋₄ alkyl, —OC(O)N(R^{d})₂, and aryloxy;
each R^{d} is independently selected from the group consisting of hydrogen, C₁₋₆ alkyl, C₂₋₆ alkenyl; C₂₋₆ alkynyl; cycloalkyl-C₀₋₆ alkyl; cycloheteroalkyl-C₀₋₆ alkyl; aryl-C₀₋₆ alkyl; and heteroaryl-C₀₋₆ alkyl, wherein the alkyl, alkenyl, alkynyl, cycloalkyl, cycloheteroalkyl, heteroaryl, and aryl in R^{d} are optionally substituted with one to four substituents each of which is independently selected from the group consisting of halo, methyl, methoxy, trifluoromethyl, trifluoromethoxy, and hydroxy;
a is an integer from 0 to 3;
m is an integer of 1 or 2;
n is an integer from 0 to 5; and
p is an integer of 0, 1, or 2.

31. The method of claim 30, wherein the fungal infection is caused by a fungus comprising Cryptococcus neoformans, Candida albicans, Aspergillus fumigatus, Trichophyton mentagrophytes, or a combination thereof.

32. The method of claim 31, wherein the subject has immunodeficiency.

33. The method of claim 32, wherein the subject has AIDS.

34. The method of claim 30, wherein Y is —NR¹R.

35. The method of claim 34, wherein R¹ and R² are hydrogen.

36. The method of claim 30, wherein R³ and R⁴ are hydrogen.

37. The method of claim 30, wherein R⁶ is hydrogen.

38. The method of claim 30, wherein a is 0.

39. The method of claim 30, wherein R⁴ is —OH.
